(19) United States
(12) Patent Application Publication
(10) Pub. No.: US 2014/0339112 A1
(43) Pub. Date: Nov. 20, 2014

(54) INJECTIBLE DRUG CARTRIDGE CONTAINER

(71) Applicants: John M. Jew, Bedminster, NJ (US); David Marxen, Bedminster, NJ (US); Joseph Rogus, Bedminster, NJ (US)

(72) Inventors: John M. Jew, Bedminster, NJ (US); David Marxen, Bedminster, NJ (US); Joseph Rogus, Bedminster, NJ (US)

(73) Assignee: NPS Pharmaceuticals ,Inc. a corporation

(21) Appl. No.: 14/342,165

(22) PCT Filed: Aug. 29, 2012
(86) PCT No.: PCT/US2012/052881

§ 371 Date: Aug. 7, 2014

Related U.S. Application Data
(60) Provisional application No. 61/528,579, filed on Aug. 29, 2011.

(51) Int. Cl. A61M 5/00; B65D 41/34

(52) U.S. Cl. CPC A61M 5/002; B65D 41/34; A61M 2205/276
USPC 206/363; 220/200; 220/288; 220/265

(57) ABSTRACT

A container comprising a tube and a cap for use in securing a carpule during shipping or storage is disclosed. The container may be configured to prevent individual components of the carpule from shifting, dislodging, or moving, particularly in response to changes in air pressure. The container may further be configured to seal and protect the carpule. A cap configured to independently secure portions of a carpule or carpule assembly is also disclosed. Additionally, embodiments of containers comprising one or more wells for securing carpules are disclosed.

### TECHNICAL FIELD

The present disclosure relates generally to packing or storage containers for injectable drug cartridges. More specifically, the present disclosure relates to containers designed to protect and secure such cartridges during shipping.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a front view of a pen-type injection device.

FIG. 2 is a partially exploded front view of the injection device of FIG. 1 shown with a carpule and holder.

FIG. 3 is an exploded view of the carpule and holder of FIG. 2.

FIG. 4 is an exploded view of the carpule and holder of FIGS. 2 and 3 shown with a container tube and cap.

FIG. 5 is an assembled view of the components of FIG. 4.

FIG. 6 is a cross-sectional view of a portion of the assembly of FIG. 5.

FIG. 7A is an exploded view of another embodiment of a carpule and a holder.

FIG. 7B is an exploded view of the carpule and holder of FIG. 7A shown with a cap.

FIG. 7C is a partial cross-sectional view of the carpule, holder, and cap of FIG. 7B in an assembled configuration.

FIG. 8 is a perspective view of a multiple well carpule container.

FIG. 9 is a top view of the multiple well carpule container of FIG. 8.

FIG. 10 is a cross-sectional view of the multiple well carpule container of FIG. 9.

FIG. 11 is a top view of another embodiment of a multiple well carpule container.

FIG. 12 is a side view of the multiple well carpule container of FIG. 11.

FIG. 13 is a partial cutaway view of another embodiment of a multiple well carpule container.

FIG. 14 is a cross-sectional view of the multiple well carpule container of FIG. 13.

### DETAILED DESCRIPTION

A cartridge may be configured to store an amount of a drug or other therapeutic agent or substance. For example, a cartridge may store one or more doses of a liquid drug, and may be used in connection with an injection device, such as a pen-type injection device. As used herein the term “carpule” (and/or the equivalent “carpoule”) refers broadly to cartridges or containers configured to store or dispense therapeutic agents, such as drugs. Such agents may be stored in solid or fluid states, including mixtures, suspensions, liquids, and so on.

A carpule may contain, for example, a liquid state drug configured to be injected through a patient's skin, such as a recombinant protein-type treatment. In some instances such drugs may degrade quickly or be difficult to store in a liquid state. Carpules may also therefore contain multiple chambers, with one or more chambers containing a solvent and one or more chambers containing freeze-dried therapeutic agents (i.e., a substance having undergone, for example, lyophilisation, lyophilization, or cryodesiccation). The carpule may (1) be used to store or ship the treatment in this more stable state and (2) be configured to aid in reconstitution of the drug at a time close to treatment.

A carpule container may be configured to stabilize and protect carpules during shipment and/or storage. For example, a carpule container may be configured to secure the components of the carpule, minimizing the chance that solvent stored therein may be allowed to spill from the carpule or interact with the freeze-dried component during shipping. In particular, components of carpules that undergo large changes in air pressure during shipment (for example, carpules shipped by airplane) may tend to move or become dislodged in response to changes in air pressure. A container may be configured to prevent such movement. Moreover, a container may be configured to protect a carpule from contamination and/or breaking.

It will be readily understood that the components of the embodiments, as generally described and illustrated in the figures herein, could be arranged and designed in a variety of configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

The phrases “in connection with,” “coupled to,” and “communication between” refer to any form of interaction between two or more entities, including mechanical, electrical, magnetic, electromagnetic, fluid, and thermal interaction. Two components may be coupled to each other even though they are not in direct contact with each other. For example, two components may be coupled to each other through an intermediate component.

The terms “proximal” and “distal” refer to opposite ends of a medical device. As used herein, the proximal end of a medical device is the end nearest a practitioner while the practitioner is using or manipulating the device, while the distal end is the opposite end. For example, the proximal end of an injection device refers to the end nearest the practitioner when the device is in use and the distal end refers to the end configured with a needle or other subcutaneous injection apparatus.

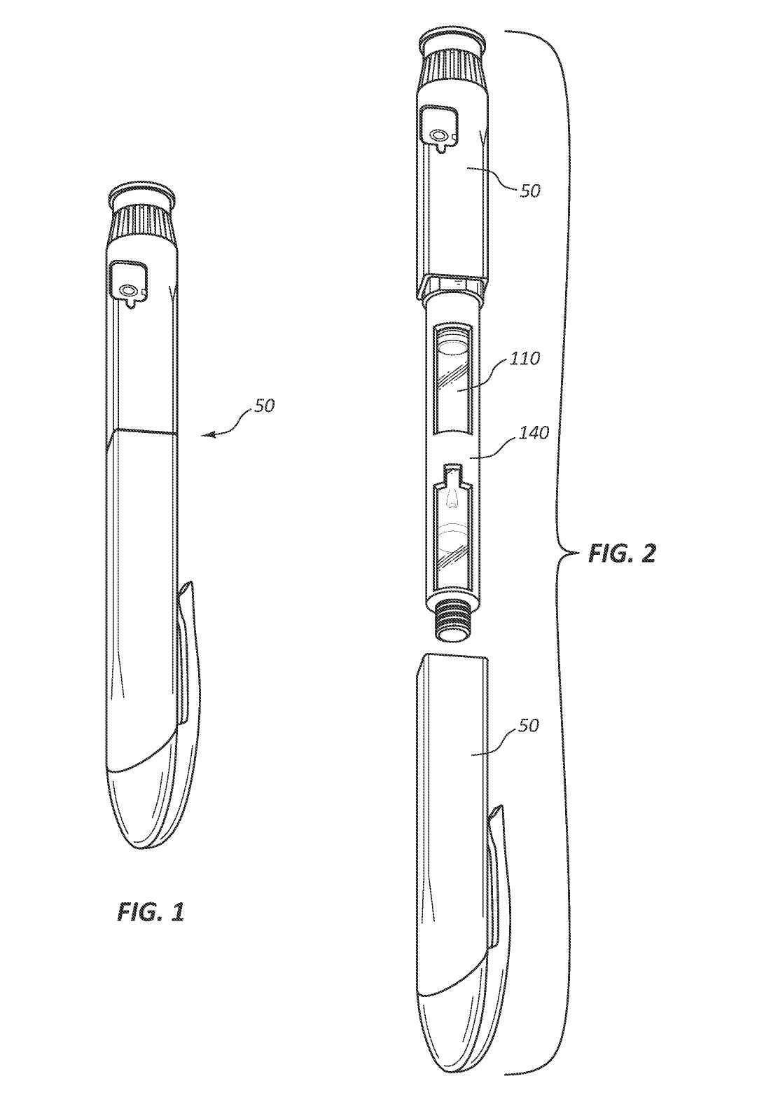

FIG. 1 is a front view of a pen-type injection device 50. Such an injection device may be configured to allow a patient to administer one or more doses of a drug to himself or herself directly. The injection device 50 may also be utilized by a health care practitioner to administer an injection to a patient. In some embodiments the injection device 50 may be configured to deliver a set number of doses of a drug through a relatively simple and error reducing process (e.g., one push button-type design).

The injection device 50 of FIG. 1 is meant to be illustrative only. It will be appreciated by one of skill in the art having the benefit of this disclosure that a wide variety of injection devices are within the scope of this disclosure. For example, any injection device that utilizes a cartridge or carpule containing drugs or other therapeutic agents is within the scope of this disclosure, whether or not the injection device resembles or shares particular components with the illustrated injection device 50.

FIG. 2 is a front view of the injection device 50 of FIG. 1, partially exploded to show how a carpule 110 may be used in connection with the injection device 50. The carpule 110 may be configured to fit directly within the injection device 50 or may be configured with a holder 140. The holder 140 may be designed to interface between the carpule 110 and the injection device 50. For example, the holder 140 may be designed to secure the carpule 110 within the injection device 50. As further detailed below, the carpule 110 may be configured to act as a storage reservoir and shipping container for drugs used in connection with the injection device 50.

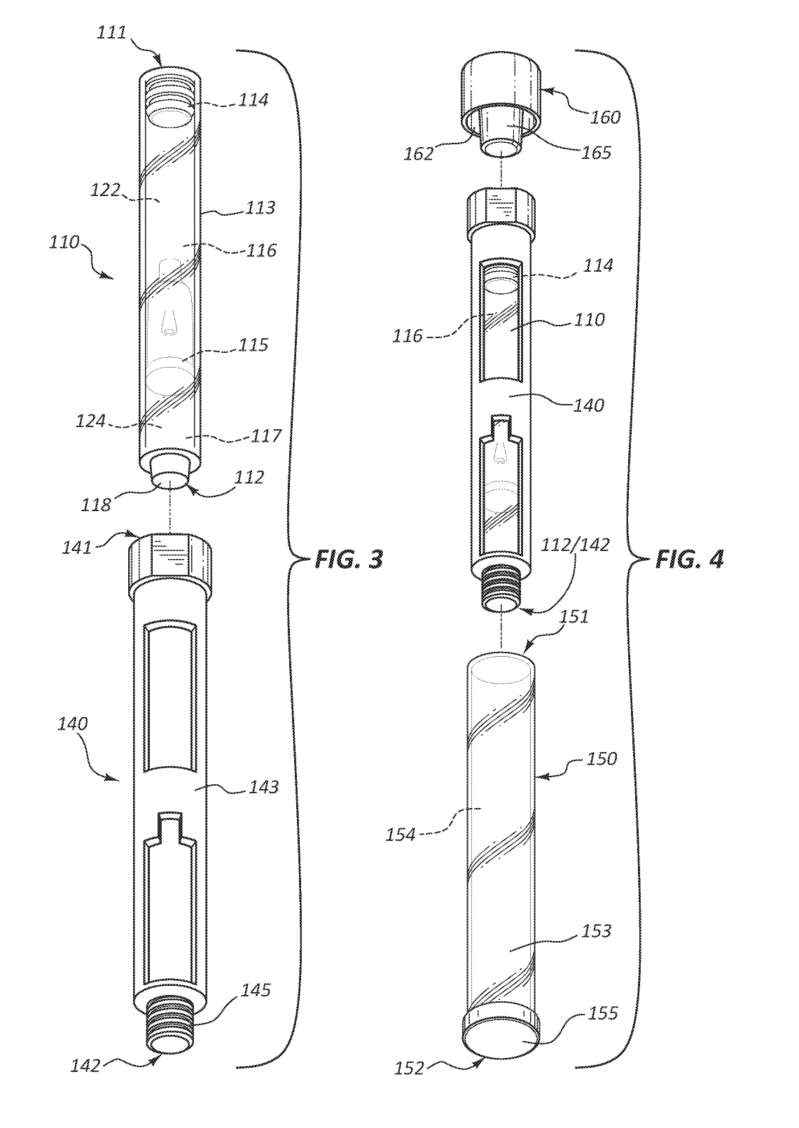

FIG. 3 is an exploded view of the carpule 110 and holder 140 of FIG. 2. The carpule 110 may define proximal 111 and distal 112 ends and comprise a body 113 disposed between the proximal 111 and distal 112 ends. In the illustrated embodiment the body 113 is generally cylindrical in shape; in other embodiments it may be any shape or form capable of comprising a reservoir for use in connection with an injection device. Thus, specific features of the geometry or shape of the body 113 may be altered from the illustrated embodiment as dictated by the injection device with which the particular carpule 110 is configured for use. The carpule body 113 may be formed of a variety of materials, including glass or plastic.

In the illustrated embodiment the body 113 of the carpule 110 is substantially hollow. An upper stopper 114 may be disposed within the body 113 adjacent the proximal end 111, and a dividing member 115 may be disposed within the body 113 between the upper stopper 114 and the distal end 112 of the carpule 110. The hollow body 113 in connection with the upper stopper 114 and the dividing member 115 may define an upper chamber 116 between the upper stopper 114 and the dividing member 115 and a lower chamber 117 between the dividing member 115 and the distal end 112 of the carpule 110. In other embodiments a carpule may have only a single chamber (i.e., no dividing member) or more than two chambers (i.e., more than one dividing member) within the body of the carpule.

In some embodiments, one of the upper 116 and lower 117 chambers may contain a solvent while the other may contain a freeze-dried drug agent. For example, in the illustrated embodiment the upper chamber 116 contains a liquid solvent 122 while the lower chamber contains a freeze-dried drug residue 124. The dividing member 115 may be configured to separate these two components during shipping and storage.

The carpule 110 may be designed such that the dividing member 115 separates the solvent 122 from the freeze-dried drug residue 124 until a user desires to reconstitute the drug. At such time, a user may breach or bypass the dividing member 115 to reconstitute the drug. In some embodiments, for example, the dividing member 115 may be a plunger or seal that may move in response to pressure exerted on the solvent 122 in the upper chamber 116. In response to this pressure the dividing member 115 may move into a position where a channel may be accessed by the solvent 122 allowing the solvent 122 to mix with the freeze-dried drug residue 124. In other embodiments the dividing member 115 may rupture in response to pressure, or the dividing member may be breached or bypassed by some other mechanism.

The carpule 110 may further comprise a septum 118 adjacent the distal end 112 of the carpule 110. The septum 118 may be configured to seal the carpule 110 when not in use and to provide access to the lower chamber 117 by components of an injection device, enabling a reconstituted drug to be dispensed from the carpule 110. For example, in some embodiments when the carpule 110 is disposed within an injection device, the proximal end of a hollow hypodermic needle (not shown) may pierce the septum 118, allowing fluid communication between the lower chamber 117 and the needle. In such an embodiment, pressure exerted by the upper stopper 114 on the reconstituted drug (after the dividing member 115 is breached) may be configured to provide doses of the drug through the hypodermic needle.

FIG. 3 further illustrates the holder 140 of FIG. 2. The holder may be configured to secure the carpule 110 within an injection device. In the illustrated embodiment, the holder 140 defines proximal 141 and distal 142 ends as well as a body 143. The body 143 may be substantially hollow, providing an inner cavity configured to receive the carpule 110. In some embodiments the distal end 142 of the holder 140 may be open, allowing components of an injection device (e.g., a hypodermic needle) to interface with the septum 118. The holder 140 may further comprise components configured to interface with the injection device, such as components configured to secure the holder 140 (and therefore the carpule 110) to the injection device. For example a holder 140 may be configured with threads 145 configured to couple the holder 140 to an injection device or a component thereof. In other embodiments, the holder 140 may simply be received within a cavity of an injection device.

FIG. 4 is a front exploded view showing how the carpule 110 and holder 140 of the preceding figures may be disposed within a container. Specifically, FIG. 4 illustrates a tube 150 having proximal 151 and distal 152 ends and a body 153.

The tube 150 may be substantially hollow, providing a receiving portion, such as inner chamber 154, that may be configured to receive a carpule 110 or a carpule 110 within a holder 140. Furthermore, the tube 150 may comprise a lower surface 155 that closes the distal end 152 of the tube 150.

The carpule 110 and holder 140 may thus be inserted into the tube 150 such that the distal ends 112, 142 of the carpule 110 and holder 140 contact the lower surface 155 of the tube 150. Thus, in some embodiments, contact between the distal ends 112, 142 of the carpule 110 and holder 140 may prevent movement (in the distal direction) of the carpule 110 and/or holder 140 with respect to the tube 150.

The tube 150 may be used in connection with a cap 160. The cap 160 may be configured to couple to the proximal end 151 of the tube 150, thus allowing the cap 160 and tube 150 to fully enclose a carpule 110 and/or holder 140 disposed within the tube 150. The cap 160 may have an inside diameter 162 configured to contact the outside diameter of the tube 150 when the cap 160 is coupled to the tube 150.

The cap 160 may further comprise a protrusion 165 coupled to the cap 160. The protrusion 165 may extend beyond the cap 160 into the tube 150 when the two components are coupled. In some embodiments the protrusion 165 may be configured to contact the upper stopper 114 of the carpule 110 when the carpule 110 is disposed within the tube 150. Contact between the protrusion 165 and the upper stopper 114 may prevent movement of the upper stopper 114 in the proximal direction. Thus, when the cap 160 is coupled to the tube 150 with the carpule 110 and/or holder 140 inside, contact between the protrusion 165 and the upper stopper 114 and contact between the distal end 112 of the carpule 110 and the lower surface 155 may prevent the upper stopper 114 from moving proximally with respect to the carpule 110 (and conversely the carpule 110 from moving distally with respect to the upper stopper 114).

An arrangement such as that shown in FIG. 4 may be utilized to prevent the upper stopper 114 from becoming dislodged during shipping of the carpule 110. In particular, in instances when the carpule 110 is shipped via airplane, ambient air pressure around the carpule 110 may drop during shipping due to the increase in altitude. This drop in air pressure may result in a relatively higher pressure within the upper chamber 116 of the carpule 110 with respect to the ambient pressure. This pressure differential, if great enough, may overcome friction between the upper stopper 114 and the carpule 110, causing the upper stopper 114 to move or become dislodged. As described above, contact between the carpule 110, upper stopper 114, tube 150, and protrusion 165 may prevent such movement.

Furthermore, the tube 150 and cap 160 may also prevent dust or other contaminants from contacting the carpule 110 during shipping or storage. The tube 150 and cap 160 may also provide additional structural integrity to the system, preventing the carpule 110 from breaking during transit.

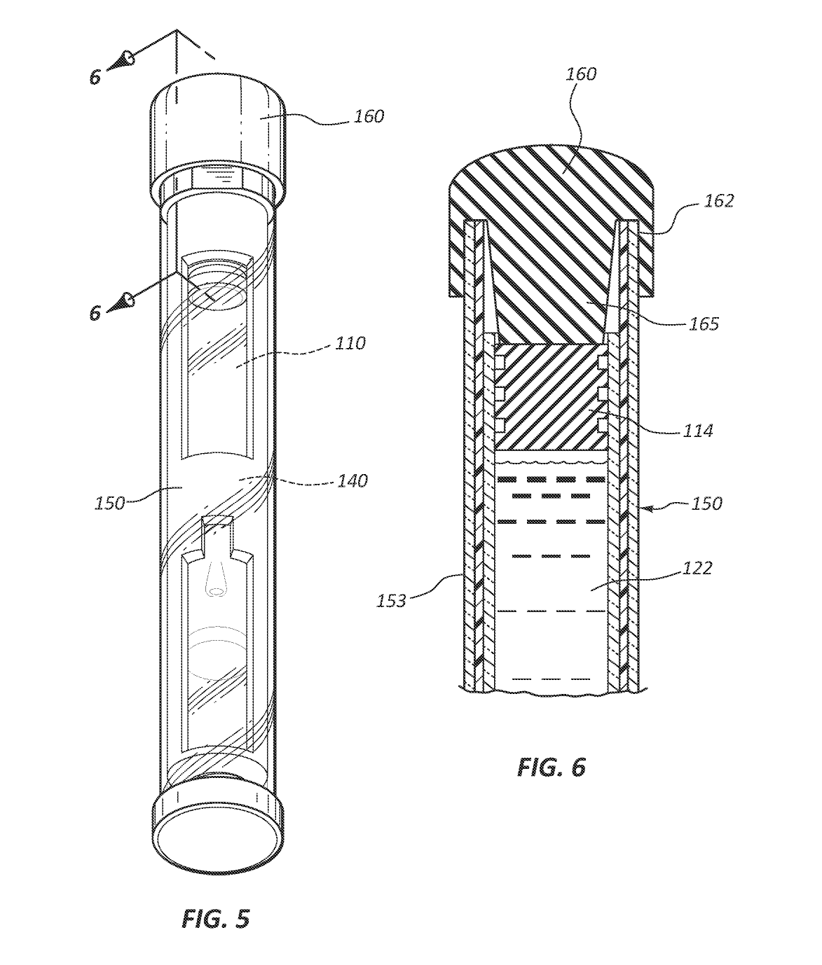

FIG. 5 is an assembled view of the components of FIG. 4, including the cap 160, the carpule 110, the holder 140, and the tube 150. FIG. 6 is a cross-sectional view, taken through line 6-6 of a portion of the assembly of FIG. 5. FIG. 6 illustrates how contact between the protrusion 165 and the upper stopper 114 prevents movement of the upper stopper 114 in response to relatively higher pressure due to the solvent 122 disposed within the carpule 110. As previously stated, the current disclosure is relevant to carpules that may have only one chamber within the carpule or carpules with two or more chambers. The protrusion may prevent movement of the upper stopper regardless of the precise configuration of chambers within the carpule.

FIG. 6 further illustrates how frictional forces between the inside diameter 162 of the cap 160 and the outside diameter of the tube body 153 may tend to keep the cap 160 in place on the tube 150. In other embodiments, either the tube body 153, the cap 160, or both may further comprise ridges or barbs configured to enhance the friction fit. Moreover, the cap 160 may also (or alternatively) be glued to the tube body 153 or welded to the tube body 153, for example by RF welding. In such embodiments the cap 160 and tube 150 may be coupled in a “break open” configuration, wherein a friable portion may be configured to allow a user to open the container. In still other embodiments the cap 160 and tube 150 may interface through threads, a twist-to-lock design, a snap-fit design, barbs, ridges, a foil or paper adhesive seal, or any other coupling means.

In any of the above embodiments, the cap 160 and tube body 153 may also be coupled in connection with a friable component (not shown) configured to break once the cap 160 is uncoupled from the tube 150. In some embodiments the friable component may be configured as a “tamper evident” feature; in other words, configured to indicate to a user whether the cap 160 has ever been removed from the tube 150. This “tamper evident” friable member may or may not be the same component as the “break open” component described above.

Furthermore, and as also detailed below, in alternative embodiments the cap may be configured to fit directly on the carpule and/or holder directly, either by a friction fit analogous to the cap/tube fit in the illustrated embodiment or by threads, twist-to-lock, or other coupling means.

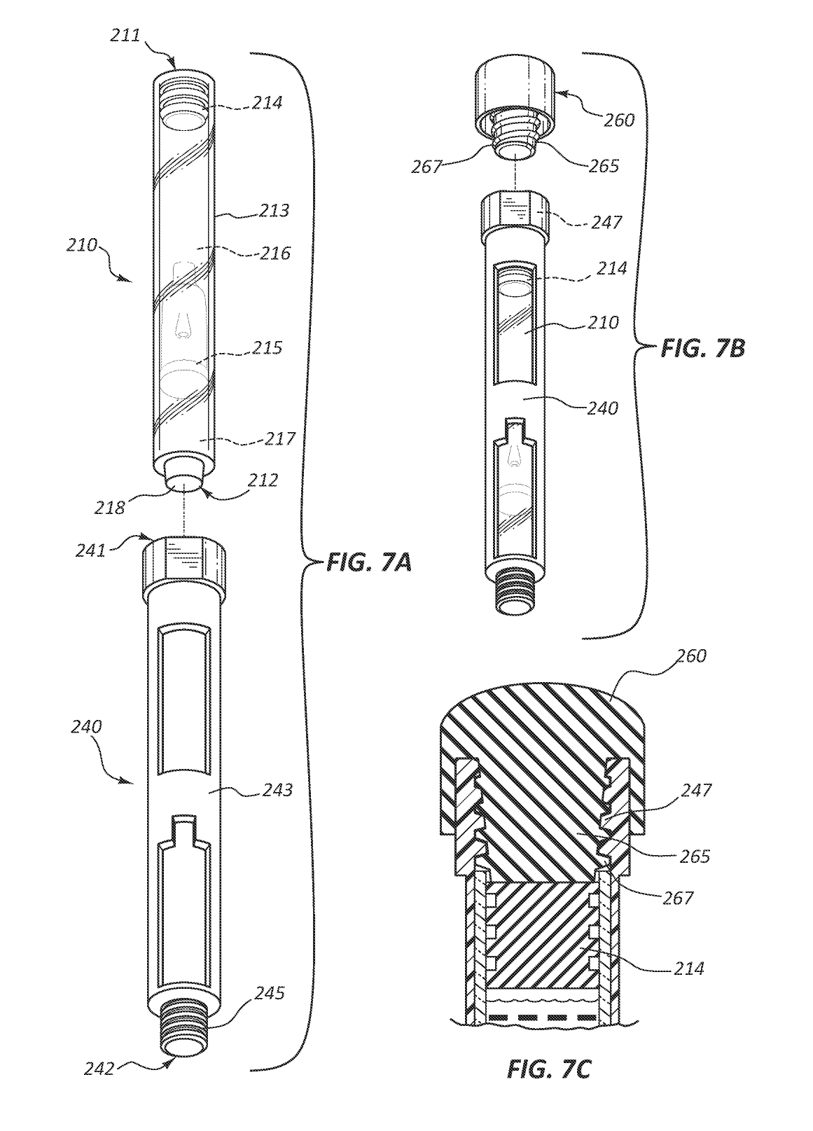

FIG. 7A is an exploded view of another embodiment of a carpule 210 and a holder 240. The embodiment of FIG. 7A, and subsequent related figures, may resemble the carpule 110 and related components of FIGS. 1-6 in some respects. It will be appreciated that all the illustrated embodiments may have analogous features. Accordingly, like features are designated with like reference numerals, with the leading digits incremented to “2.” (For instance, the carpule is designated “110” in FIG. 3 and an analogous carpule is designated as “210” in FIG. 7A.) Relevant disclosure set forth above regarding similarly identified features thus may not be repeated hereafter. Moreover, specific features of the carpule, holder, containers, and other related components shown in FIG. 7A may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the embodiment of FIG. 7A. Any suitable combination of the features, and variations of the same, described with respect to the embodiment illustrated in FIGS. 1-6 can be employed with the embodiment of FIG. 7A, and vice versa. This pattern of disclosure applies equally to further embodiments depicted in subsequent figures and described hereafter.

The embodiment of FIG. 7A includes a carpule 210 comprising a body 213 extending from a proximal end 211 to a distal end 212. A septum 218 is disposed adjacent the distal end 212 of the carpule 210. Further the carpule 210 defines an upper chamber 216 and a lower chamber 217 which are separated by a dividing member 215. Finally, the carpule 210 comprises an upper stopper 214 disposed adjacent the proximal end 211.

FIG. 7A further illustrates a holder 240. The holder 240 comprises a body 243 extending between a proximal end 241 and a distal end 242. The holder 240 may be configured to interface with injection devices or other components through elements such as interface threads 245. The carpule 210 may be configured to be disposed within the holder 240, as indicated in FIGS. 7A and 7B.

FIG. 7B is an exploded view of the carpule 210 and holder 240 of FIG. 7A shown with a cap 260. The cap 260 may be configured to be coupled to the holder 240 adjacent the proximal end 241 of the holder 240. In the illustrated embodiment, the cap 260 comprises cap threads 267 on the outside diameter of a protrusion 265. These cap threads 267 may be configured to mate with holder threads 247 disposed on an inside diameter of the holder 240. In other embodiments, other coupling mechanisms such as snap fits, ribs, interference fits, friction fits, pins, and so forth may be configured to couple the cap 260 to the holder 240. The outside diameter of the cap 260 may comprise ridges, angles, or other surfaces configured to facilitate twisting of the cap 260 by a user. In some embodiments, the outside profile of the cap 260 may be rectangular or comprise other shapes having corners which may facilitate gripping of the cap 260.

In the illustrated embodiment, the protrusion 265 is configured to extend from the cap 260 and contact the upper stopper 214 of the carpule 210 when the cap 260 is coupled to the holder 240. This contact may prevent the upper stopper 214 from moving proximally with respect to the carpule 210. FIG. 7C is a partial cross-sectional view of the carpule 210, holder 240, and cap 260 of FIG. 7B in an assembled configuration. The interaction between the protrusion 265 and the upper stopper 214 is shown in this view. Also, the mating interaction of the cap threads 267 and the holder threads 247, coupling the cap 260 to the holder 240, are shown. Thus, in some embodiments, a cap, such as cap 260, may be configured to couple directly to a holder, such as holder 240, to secure an upper stopper, such as upper stopper 214, within a carpule. Again, in other embodiments, a cap 260 or similar element may couple directly to the carpule 210 or to another intermediate component to similarly arrest the proximal movement of the upper stopper 214 with respect to the carpule 210.

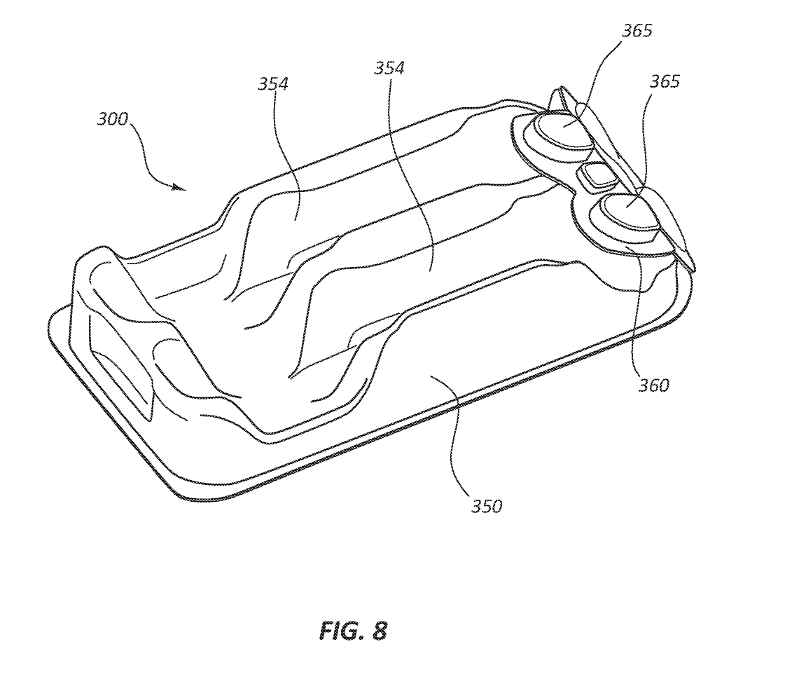

In some embodiments, a carpule container may be configured with multiple wells, or portions configured to receive a carpule or multiple carpules. FIG. 8 is a perspective view of a multiple well carpule container 300. A multiple well carpule container 300 may comprise a body member 350 having one or more receiving portions such as wells 354 disposed therein. The wells 354 may each be configured to retain a carpule. For example, in some embodiments, the wells 354 may comprise ridges or other features configured to allow a carpule to “snap” into the well 354 and be secured through an interference fit. In other embodiments, a lip or other component may be configured to retain the carpules within the wells 354.

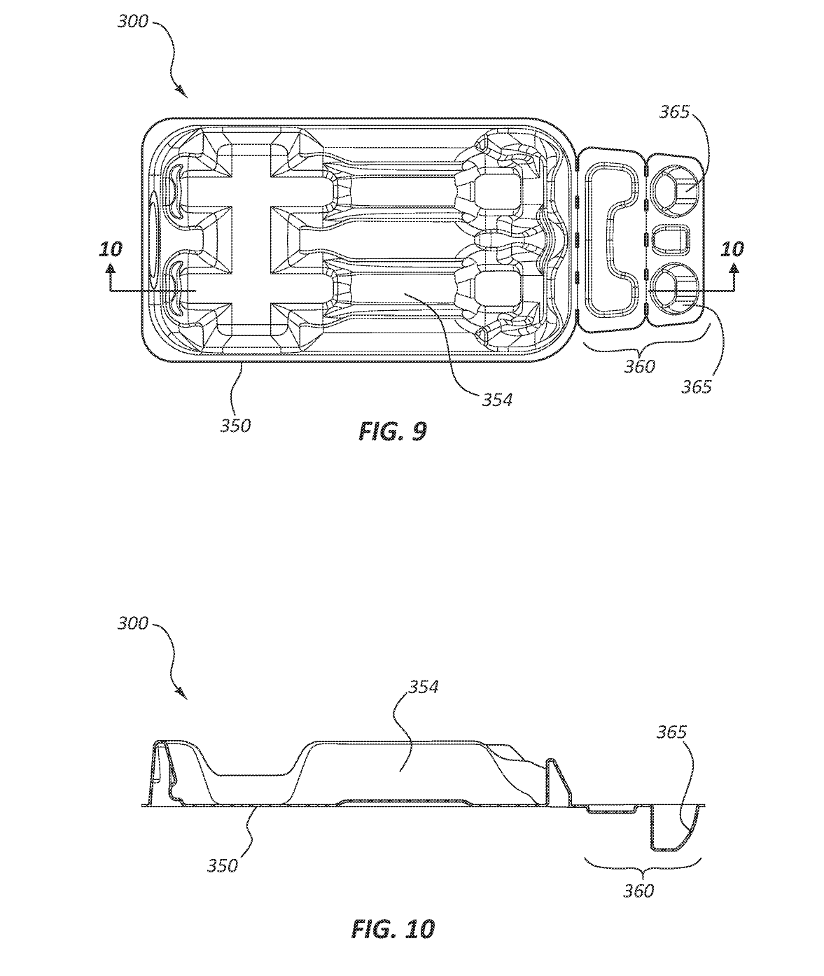

The container 300 may further comprise a retaining portion 360 comprising one or more protrusions 365. Each protrusion 365 may be associated with a well 354 of the container 300. In some embodiments the retaining portion 360 may comprise a flap configured to fold into position, while in others the protrusions 365 may be directly coupled to the body member 350. FIG. 9—a top view of the multiple well carpule container 300 of FIG. 8—illustrates how the retaining portion 360 may be configured to fold into position with respect to the body member 350 and wells 354. FIG. 10—a cross-sectional view taken through plane 10-10 of the multiple well carpule container 300—further illustrates this relationship. Referring to FIGS. 8-10, the body member 350, retaining portion 360, and protrusions 365 may each be formed of separate components or may be integrally formed. In some embodiments the container 300 may be formed by vacuum molding a thermoplastic or other material.

The wells 354 of the container 300 may be configured to receive a carpule directly (such as carpule 110 of FIG. 3), a carpule disposed within a holder (such as holder 140 of FIG. 4), or a carpule in connection with another intermediate component. Regardless, the protrusions 365 may be configured to contact an upper stopper (such as upper stopper 114 of FIG. 3) or other component of a carpule within the wells 354. Thus, the protrusions 365 may be configured to interact with, and arrest the relative movement of, portions of a carpule or carpule assembly.

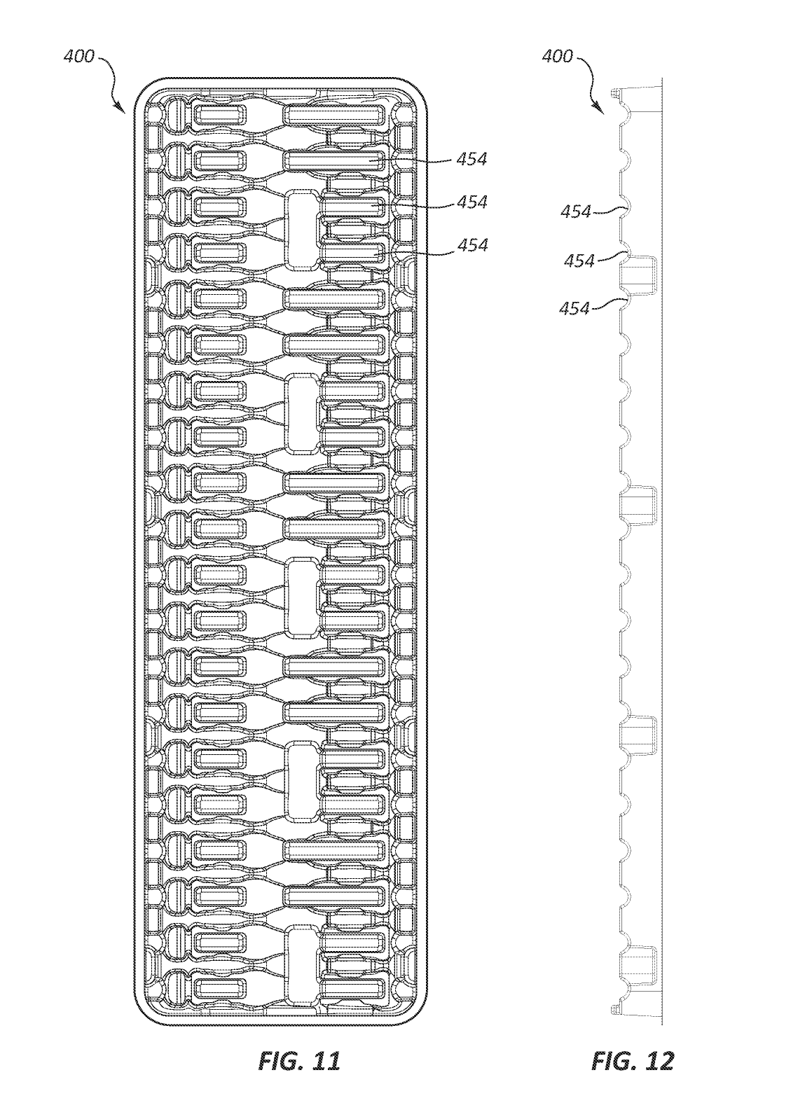

The container 300 of FIG. 8 is configured with two wells 354, and is thus configured to retain two carpules. FIG. 11 is a top view of another embodiment of a multiple well carpule container 400, and FIG. 12 is a side view of the same. In the embodiment of FIGS. 11 and 12, the container 400 is configured with twenty receiving portions, wells 454, each configured to retain a carpule. In other embodiments a container may be configured with any number of wells. For example, some containers may comprise from about two wells to about fifty wells, from about two wells to about thirty wells, and so forth. Furthermore, containers, such as those of FIGS. 8-12, may be configured with a single well. Additionally, as compared with the container 300 of FIGS. 8-10, the container 400 of FIGS. 11-12 has no foldable retaining portion 360. Rather, the features of the wells 454 are directly molded and configured to retain carpules and secure an upper stopper, or other portions, of a carpule.

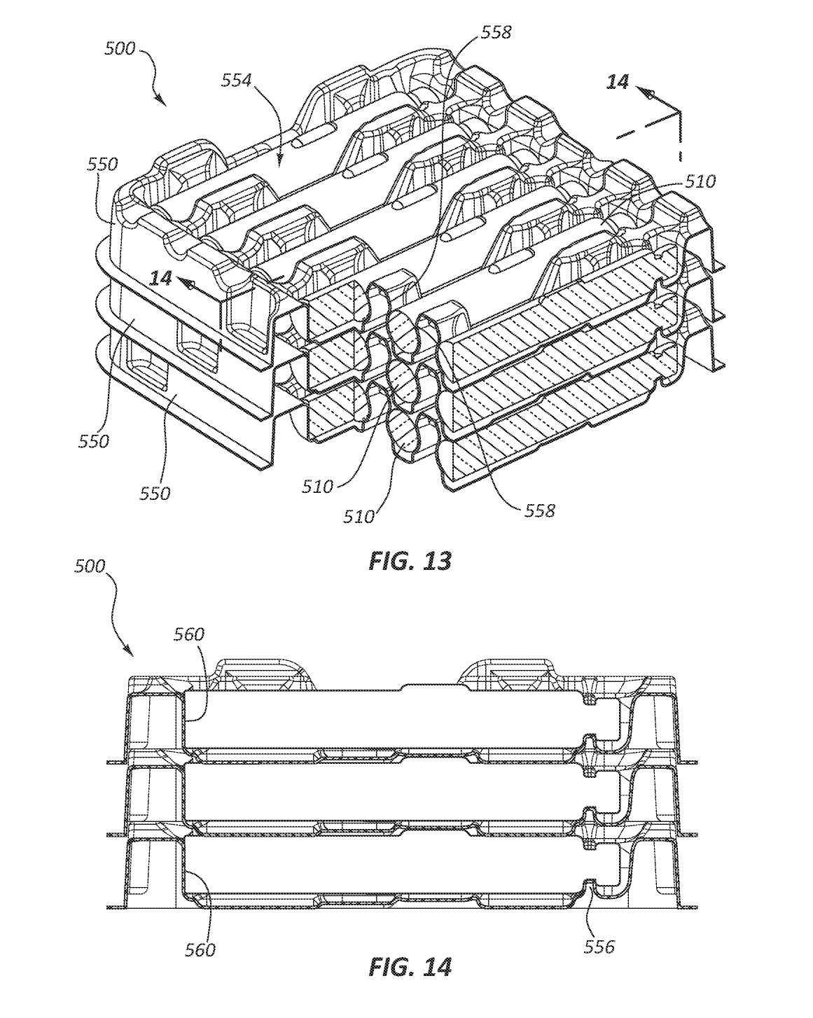

FIGS. 13-14 are views of another embodiment of a carpule container 500. Specifically, FIG. 13 is a partial cutaway view of the multiple well carpule container 500, and FIG. 14 is a cross-sectional view of the multiple well carpule container 500 taken through plane 14-14. The container 500 comprises multiple body members 550, which may be configured to be stacked on each other. Each body member 550 comprises receiving portions, such as wells 554 configured to retain carpules 510. Though carpules 510 are illustrated in FIGS. 13-14, this, as well as any embodiment, could be configured to retain carpules 510 within holders (such as holder 140 of FIG. 3) or carpules 510 within other components. Any number of body members 550 may be configured to be stacked together.

The container 500 comprises a retaining portion 560 integrally formed with the body member 550. The retaining portion 560 may be configured to abut an end of the carpule 510 and prevent dislodgement of a portion of the carpule 510 (such as the upper stopper 114 of FIG. 3). In the embodiment of FIGS. 13-14 the retaining portion 560 directly abuts the end of the carpule 510, while in other embodiments a protrusion or other feature may be configured to extend from the retaining portion 560 into contact with the carpule 510 or any component thereof. The body member 550 may further comprise support portions 556 configured to interact or mate with portions of the carpule 510. These support portions 556 may be configured to support, retain, align, position, or otherwise interact with the carpules 510. The body member 550 also comprises retaining ridges 558 configured to retain the carpules 510 within the wells 554. In the illustrated embodiment these ridges create an interference-type “snap” fit between the body member 550 and the carpule 510.

Any of the containers (such as 300, 400, and 500 of FIGS. 8-14), caps (such as 160 and 260 of FIGS. 4-7C), or tubes (such as 150 of FIGS. 4-6) may be configured to store, retain, or secure one or more carpules in various ways. For example, these components may be configured to prevent movement or dislodgement of one or more stoppers (such as upper stopper 114 of FIG. 3) within a carpule. These components may alternatively or additionally be configured to protect the carpules during shipping or delivery, prevent unwanted contact between the carpule and foreign surfaces, and provide a packaging assembly for multiple carpules. Some containers may be configured to hold a large number of carpules for bulk shipment of carpules, while others may be configured to hold a smaller number for retail sale. Finally, any of these components or embodiments may be configured to interact with carpules of various designs, shapes, or sizes.

The examples and embodiments disclosed herein are to be construed as merely illustrative and exemplary, and not a limitation of the scope of the present disclosure in any way. It will be apparent to those having skill in the art with the aid of the present disclosure that changes may be made to the details of the above-described embodiments without departing from the underlying principles of the disclosure herein.

1. A cap configured to secure a portion of a carpule, the cap comprising:
a body; and
a protrusion configured to contact a portion of a carpule such that the protrusion restricts movement of the portion of the carpule.

2. The cap of claim 1, wherein the cap couples to the carpule directly.

3. The cap of claim 1, wherein the cap couples to a holder coupled to the carpule.

4. The cap of claim 3, wherein the cap couples to the holder by threads.

5. The cap of claim 1, further comprising a friable or tamper evident removable feature disposed between the cap and carpule.

6. A carpule container comprising:
a receiving portion configured to receive a carpule; and
a retaining portion, the retaining portion configured to contact a portion of a carpule disposed within the receiving portion such that the retaining portion restricts movement of the portion of the carpule.

7. The carpule container of claim 6, further comprising a body member formed such that the receiving portion comprises a well within the body member.

8. The carpule container of claim 7, wherein the retaining portion further comprises a protrusion configured to contact the portion of the carpule.

9. The carpule container of claim 8, wherein the body member, the retaining portion, and the protrusion are integrally formed.

10. The carpule container of claim 9, wherein the carpule is configured to snap into the receiving portion.

11. The carpule container of claim 9, further comprising a plurality of receiving portions and a plurality of retaining portions.

12. The carpule container of claim 6, wherein the retaining portion is configured to contact a holder configured to retain a carpule.

13. The carpule container of claim 6, further comprising:
a tube having a first open end and a second closed end, the tube formed such that the receiving portion comprises an interior portion of the tube; and
wherein the retaining portion comprises:
a cap configured to couple to the first open end of the tube; and
a protrusion coupled to the cap, the protrusion configured to contact the portion of the carpule.

14. The carpule container of claim 13, wherein the cap couples to the tube by a friction fit.

15. The carpule container of claim 13, wherein the cap couples to the tube by threads.

16. The carpule container of claim 13, wherein the cap couples to the tube by a twist-to-lock mechanism.

17. The carpule container of claim 13, wherein the cap couples to the tube by a glued fit and the container further comprises a friable or tamper evident removable feature for opening.

18. The carpule container of claim 13, wherein the cap couples to the tube by a welded fit and the container further comprises a friable or tamper evident removable feature for opening.

19. The carpule container of claim 13, wherein the cap couples to the tube by a locking or snap fit.

20. The carpule container of claim 6, wherein the container further comprises a friable or tamper evident removable feature.
